(19) United States
(12) Patent Application Publication
(10) Pub. No.: US 2013/0268283 A1
(43) Pub. Date: Oct. 10, 2013

(54) Process to Streamline Workflow for Continuous Monitoring of a Patient

(71) Applicants: John Raymond Vann, Auburn, NY (US); Robert Paul Wilmington, Vancouver, WA (US); Edward Imboden, Syracuse, NY (US)

(72) Inventors: John Raymond Vann, Auburn, NY (US); Robert Paul Wilmington, Vancouver, WA (US); Edward Imboden, Syracuse, NY (US)

(73) Assignee: WELCH ALLYN, INC., Skaneateles Falls, NY (US)

(21) Appl. No.: 13/440,361

(22) Filed: Apr. 5, 2012

(51) Int. Cl. G06Q 50/22

(52) U.S. Cl. 705/2

(57) ABSTRACT

A method for displaying patient data includes receiving physiological data from a medical monitoring device. The medical monitoring device obtains the physiological data from a patient. A determination is made as to whether patient identification information and room location information have been received for the patient. When it is determined that both the patient identification information and the room location information for the patient have not been received, the physiological data is displayed at a temporary location on a display screen of a medical display device.

### BACKGROUND

In a medical setting, monitoring devices are typically used to monitor physiological data from patients. The monitoring devices receive physiological data from patient sensor devices that are attached to the patients. The physiological data from the monitoring devices may be sent to a centrally located display device, for example a central monitoring display device located at or near a nurse's station, whereby clinicians may view physiological data from multiple patients at the centrally located location.

For security purposes, patients need to be identified so that the correct physiological data is associated with the correct patient. However, when a patient enters a medical setting under an emergency situation, there may not be enough time to completely identify the patient.

### SUMMARY

Embodiments of the disclosure are directed to systems and methods for displaying patient data. Physiological data is received from a medical monitoring device. The medical monitoring device obtains the physiological data from a patient. A determination is made as to whether patient identification information and room location information have been received for the patient. When it is determined that both the patient identification information and the room location information for the patient have not been received, the physiological data is displayed at a temporary location on a display screen of a medical display device.

The details of one or more techniques are set forth in the accompanying drawings and the description below. Other features, objects, and advantages of these techniques will be apparent from the description, drawings, and claims.

### DESCRIPTION OF THE DRAWINGS

FIG. 1 shows an example system that supports a workflow for a process to streamline continuous monitoring of a patient.

FIG. 2 shows an example user interface of the central monitoring station of FIG. 1.

FIG. 3 shows the waiting area of FIG. 1 in more detail.

FIG. 4 shows the example user interface of FIG. 2 with a larger waiting area.

FIG. 5 shows the compressed tile format of FIG. 3 in more detail.

FIG. 6 shows a normal tile format.

FIG. 7 shows an example parameter display area for a compressed tile associated with a continuous device.

FIG. 8 shows an example parameter display area for a compressed tile representing episodic readings only.

FIG. 9 shows an example screen shot of a dialog box for entering data into a compressed tile in a waiting area.

FIG. 10 shows an example screen shot of a device detail screen.

FIG. 11 shows an example flowchart for a method for entering patient data into a medical display device.

FIG. 12 illustrates example physical components of the central monitoring station of FIG. 1.

### DETAILED DESCRIPTION

The present disclosure is directed to systems and methods for a medical workflow that streamlines a process for continuous monitoring of physiological data of a patient. The systems and methods are directed to a waiting area on a display screen of a centrally located patient monitoring device, also known as a central station. The central station provides a display of a plurality of display tiles, each display tile displaying physiological data for one of a plurality of patients. The waiting area is a tile display area located in a section of the display screen. The waiting area provides one or more compressed display tiles, each compressed display tile corresponding to a patient for which physiological data is being received but for which the patient has not been completely identified.

In examples described herein, physiological data received from patients that have not been completely identified is continually displayed in the compressed display tiles in the waiting area. The physiological data is also sent to a central storage site, for example an electronic medical records (EMR) server computer. When sufficient identification information is received at the central station to completely identify the patient, the compressed tile for the patient is removed from the waiting area, expanded to include the size and format of a normal display tile and displayed as a normal display tile on the display screen of the central station.

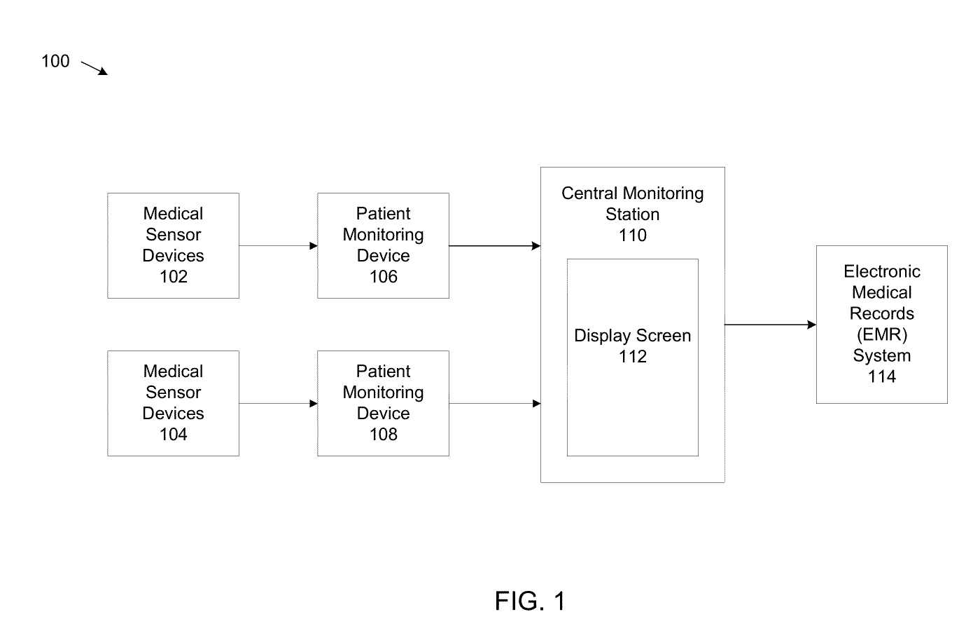

FIG. 1 shows an example system 100 that supports a workflow for a process to streamline continuous monitoring of a patient. The example system 100 includes medical sensor devices 102 and 104, patient monitoring devices 106 and 108, central monitoring station 110 and EMR system 114. More or fewer patient sensor devices and patient monitoring devices may be used.

In the example system 100, medical sensor devices 102 are attached to a patient requiring continuous monitoring, for example a surgical patient or a post-surgical patient in an intensive care unit. The example patient monitoring device 106 is a continuous monitoring device, receiving continuous physiological data from medical sensor devices 102. In this example, continuous physiological data refers to physiological data obtained periodically at short intervals. Certain physiological data, for example SPO2 and pulse rate, may be obtained in millisecond intervals. Other physiological data, for example non-invasive blood pressure (NIBP) may be obtained on a periodical basis, but at longer intervals, for example every few minutes. An example continuous monitoring device is the Welch Allyn 1500 Patient Monitor from Welch Allyn, Inc. of Skaneateles Falls, N.Y.

In the example system 100, medical sensor devices 104 are attached to a patient receiving non-continuous episodic monitoring. For example, a patient monitoring device 108 may include some or all of the medical sensor devices 104, for example a thermometer, a blood pressure cuff and an SPO2 sensor. The patient monitoring device 108 may be a portable vital signs device administered by a clinician on an as needs basis. An example vital signs device is the Connex® Vital Signs Monitor from Welch Allyn, Inc. of Skaneateles Falls, N.Y.

When the patient is completely identified, the example central monitoring station 110 receives physiological data from patient monitoring device 106 and from patient monitoring device 108 and displays the physiological data on example display screen 112. In addition, the central monitoring station 110 sends physiological data received from patient monitoring devices 106 and 108 to EMR system 114.

In this disclosure, completely identifying a patient comprises providing at least three items of identifying information. One item of identifying information includes an identifier for a medical sensor device that is sensing physiological data for the patient. In examples, the identification for the medical sensor device is automatically provided to the central station when the medical sensor device sends physiological data for the patent to the central station.

A second item of identifying information is a location for the medical sensor device, for example a room number for the patient. Other examples of location information may include a bed number, a location on a hospital grid, etc. Additional examples of location information are possible. In some examples, more than one type of location information may be needed, for example a room number and a bed number.

A third item of identifying information is an identifier for the patient, for example a patient ID. In examples, the patient ID may be obtained from scanning a wristband attached to the wrist of the patient. At some time during the emergency situation, the wristband may be attached to the wrist of the patient, for example when the wristband is completed and the medical status of the patient is such that the wristband may be attached.

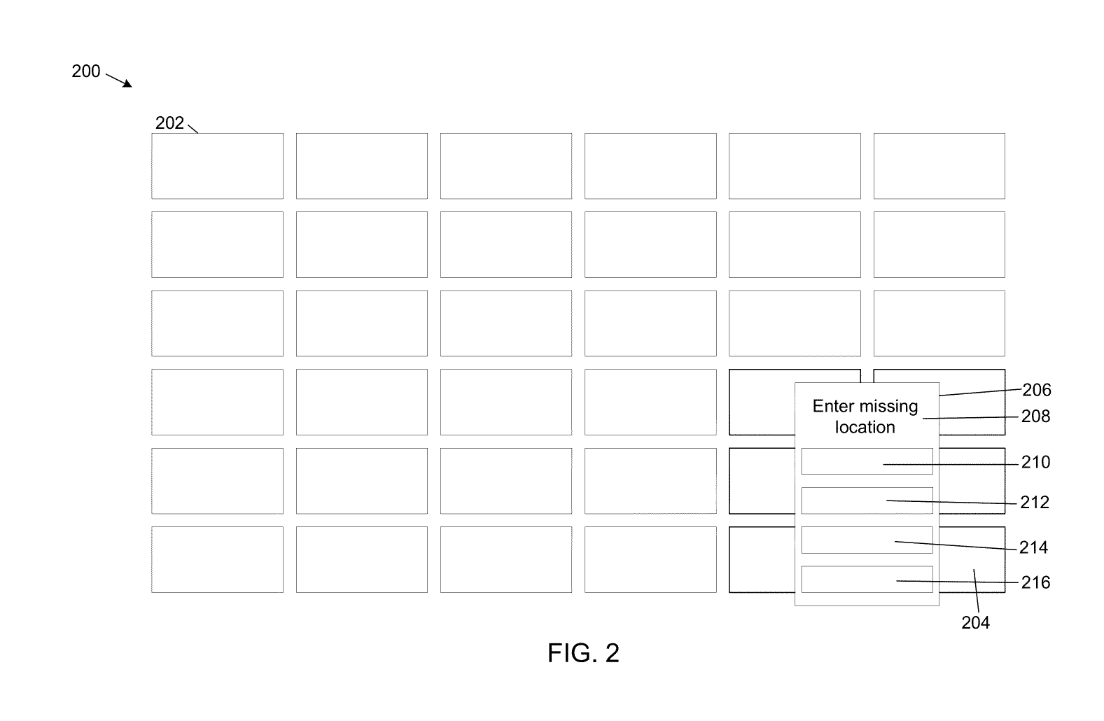

FIG. 2 shows an example user interface 200 of display screen 112. The example user interface 200 includes a rendering of 36 display tiles. In FIG. 2, the 36 display tiles are rendered in six rows of six tiles each. The example first tile on the first row is designated tile 202 and the last example tile is designated tile 204. The maximum number of tiles displayed typically corresponds to a number of patients in an area centrally located around a nurse's station where the central station is located. In examples, more or fewer display tiles may be displayed. Another common maximum number of display tiles is 48 tiles. When more than 48 display tiles are displayed on display screen 112, the display tiles may be too small to be viewed at a normal viewing distance, unless a screen of larger dimensions is used.

The example user interface 200 of display screen 112 includes a waiting area 206. The example waiting area 206 includes four compressed display tiles 210, 212, 214, and 216. The waiting area 206 also includes a message area 208, in this example displaying the phrase “Enter missing location.” The waiting area 206 is displayed on the user interface 200 of display screen 112 when there is at least one compressed display tile, indicating that physiological data is being received for a patient, but that the patient has not been completely identified. The waiting area 206 is displayed such that the waiting area 206 does not completely obscure any normal display tile on the user interface 200. As shown in FIG. 2, the waiting area 206 only partially obscures the display tiles below the waiting area 206.

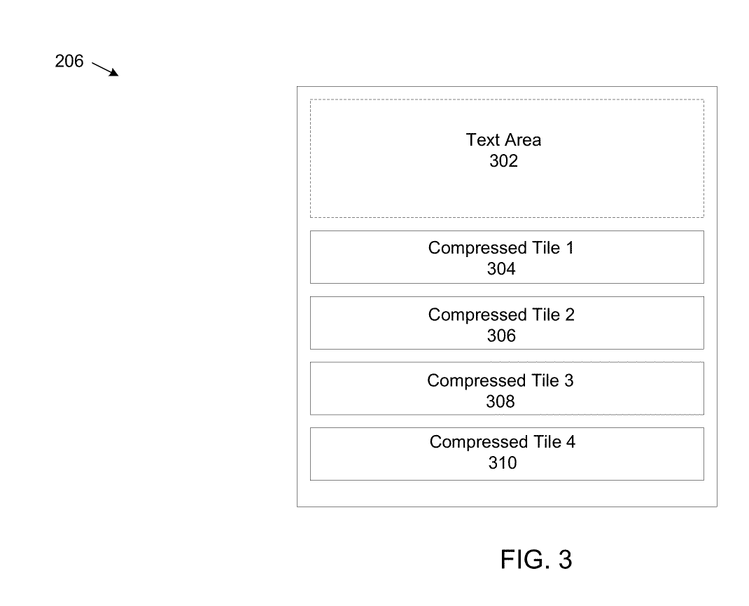

FIG. 3 shows the waiting area 206 in more detail. The example waiting area 206 includes a text area 302, and compressed tiles 304, 306, 308 and 310. The example text area 302 provides an area for displaying messages relating to the waiting area 206. An example text message is “Enter missing location.” As another example, when a manual tile layout mode is used, an example text message is “Enter missing location, then drag tile into position.” The compressed tiles 304, 306, 308, 310 provide physiological data for a patient in a compressed format.

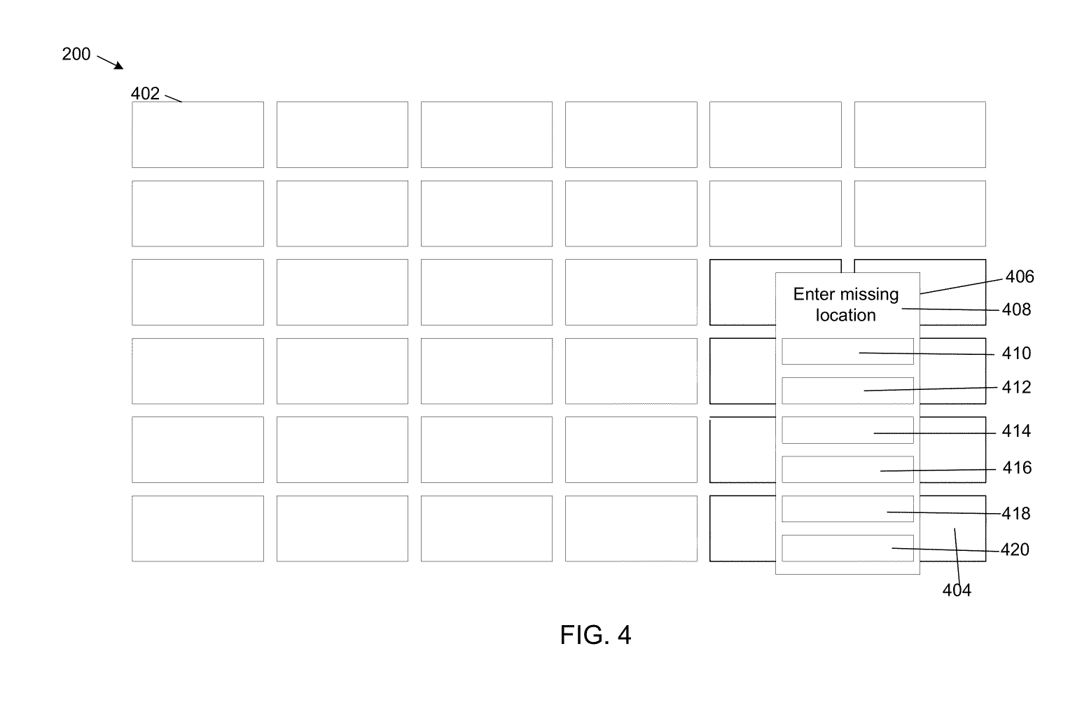

In general, the physiological data displayed in the compressed tiles 304, 306, 308, 310 is displayed in a smaller font than for normal display tiles and, typically, only a subset of the data of normal tiles is displayed as compressed tiles. More or fewer than four compressed tiles may be included in the waiting area 206. As more compressed tiles are assigned to patients, the waiting area grows higher in a direction towards the top of the user interface 200, as shown in FIG. 4. When the waiting area 206 grows to the height of the user interface 200 and a new compressed tile is added to the waiting area 206, a scroll bar (not shown) is added to the waiting area 206.

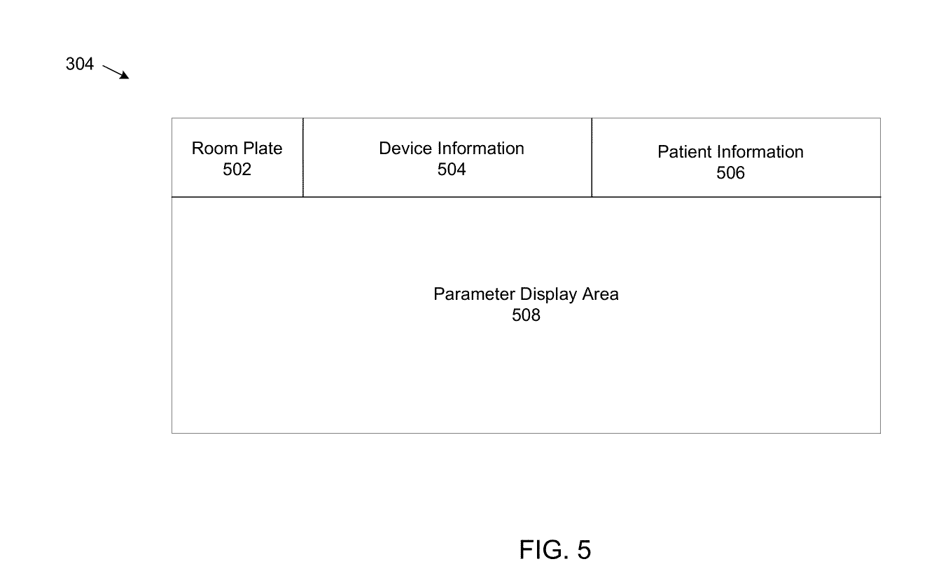

FIG. 5 shows the compressed tile 304 in more detail. The example compressed tile 304 includes a room plate 502, device information 504, patient information 506 and a parameter display area 508. The example room plate 502 displays a room number and typically a bed number for a patient. The device information 504 includes a name of the medical device attached to the patient. The example patient information 506 includes the name of the patient, the sex of the patient, and typically a patient ID. The example parameter display area 508 displays physiological parameters for the patient. Because tile 304 is a compressed tile in the waiting area 206, at least one of the room plate 502, device information 504 and patient information 506 have not been provided. Because the device information 504 is known, typically either the location of the patient, as displayed in the room plate 502 and the patient information 506 are not known.

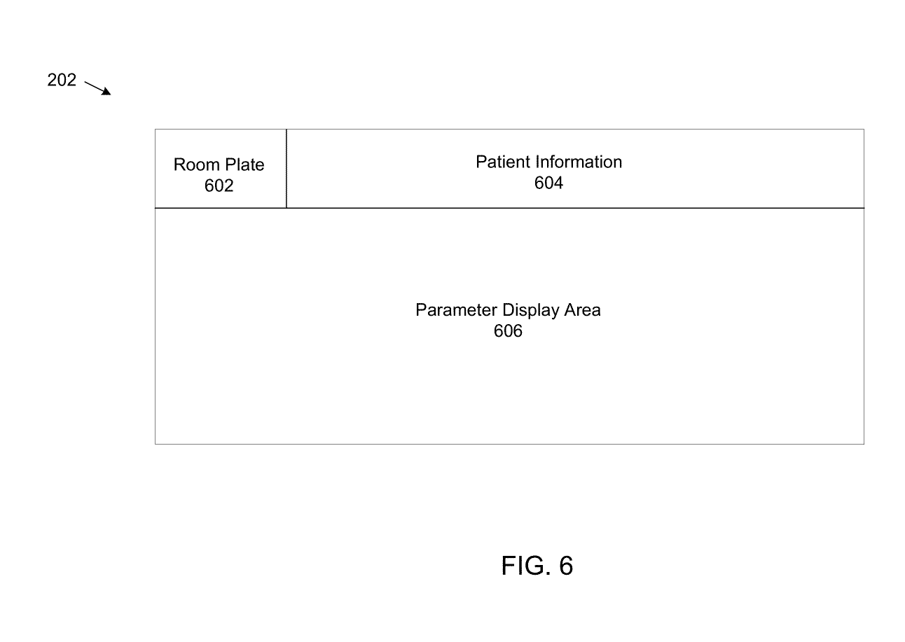

FIG. 6 shows an example normal display tile 202 in more detail. The example display tile 202 includes a room plate 602, patient information area 604 and parameter display area 606. The normal display tile 202 has the same structure as the compressed display tile, except that the device information 504 is not displayed. When all three required fields, device ID, location and patient information have been provided, the compressed display tile 304 is transformed into a normal display tile and the device information 504 is no longer displayed.

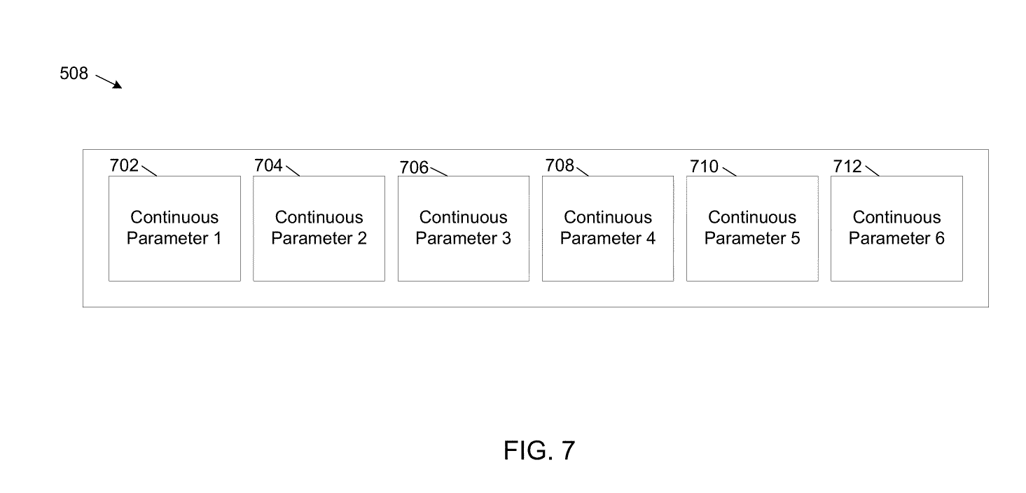

FIG. 7 shows an example parameter display area 508 for a compressed tile associated with a continuous device. A continuous device is a medical sensor device that provides continuous physiological data to the central monitoring station 110. In this disclosure, continuous physiological data is periodic physiological data having intervals in the order of milliseconds. The example parameter display area 508 displays physiological data for six continuous parameters 702, 704, 706, 708, 710 and 712. Examples of continuous parameters are oxygen saturation, pulse rate, respiration rate, end-tidal carbon dioxide and hemoglobin. Other continuous parameters are possible.

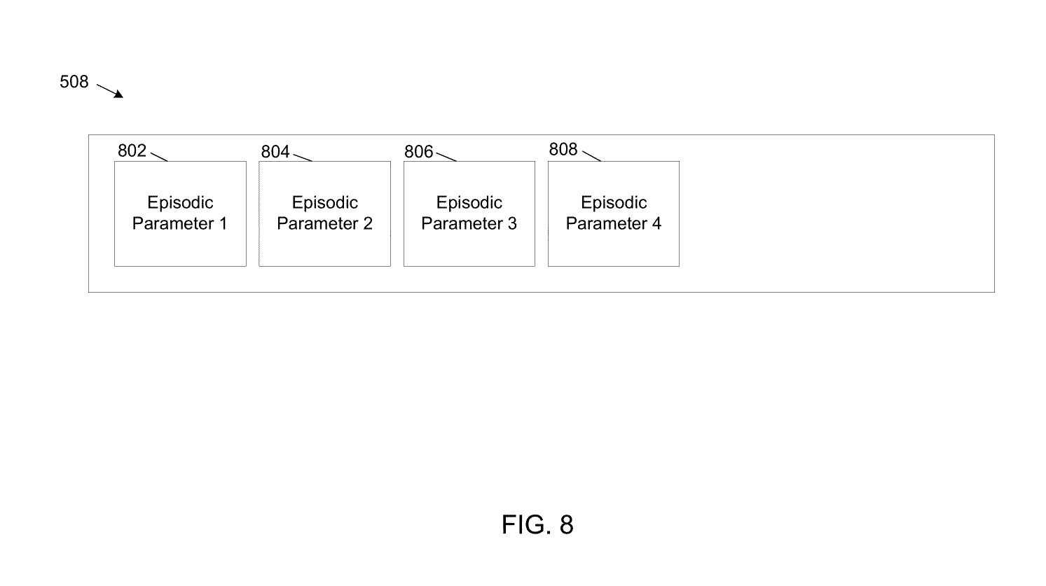

FIG. 8 shows an example parameter display area 508 for a compressed tile representing episodic readings only. Episodic parameters represent physiological data that is obtained at random intervals, for example periodically during a nurse's shift. The example parameter display area 508 displays physiological data for four episodic parameters 802, 804, 806 and 808. Examples of episodic parameters are non-invasive blood pressure, temperature, pulse rate and oxygen saturation. Other episodic parameters are possible.

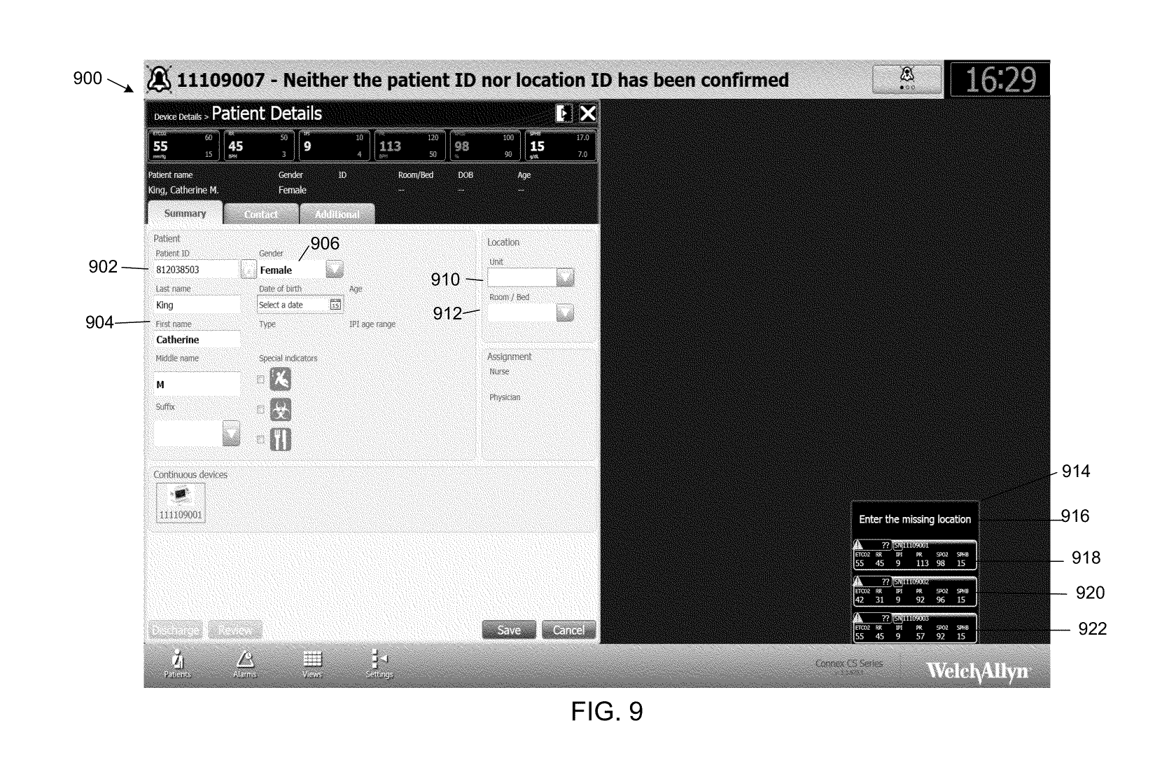

FIG. 9 shows an example screen shot 900 of a dialog box for entering data into a compressed tile in a waiting area. The data that may be entered includes a patient ID 902, a patient name 904, a patient's sex 906 and location information including a care unit 910 and a room/bed number 912. A care unit is a section of a building. The care unit may be a single floor of the building or may be on more than one floor of the building, or on a wing of a floor. Some examples of care unit 910 are an intensive care unit (ICU) and a continuous care unit (CCU). Other common names for a care unit are “MedSurge”, “Neuro”, “Pediatrics” and “Oncology.” Other examples of care units are possible. In examples the example dialog box is displayed when a user clicks on a compressed tile in the waiting area. In examples, patient information, including the patient ID 902, may be obtained by scanning a patient's identification wristband.

FIG. 9 also shows a reproduction of a waiting area 914. The waiting area 914 displays an example message 916 “Enter the missing location.” The example message 916 provides an alert to a clinician that missing data is to entered for the compressed tiles in the waiting area 914. The waiting area 914 includes the compressed tiles 918, 920 and 922. In FIG. 9, the physiological and other data in compressed tile 918 corresponds to the data being entered into the dialog box shown in screen shot 900. In this example, the dialog box in screen shot 900 is displayed when a user clicks on compressed tile 918.

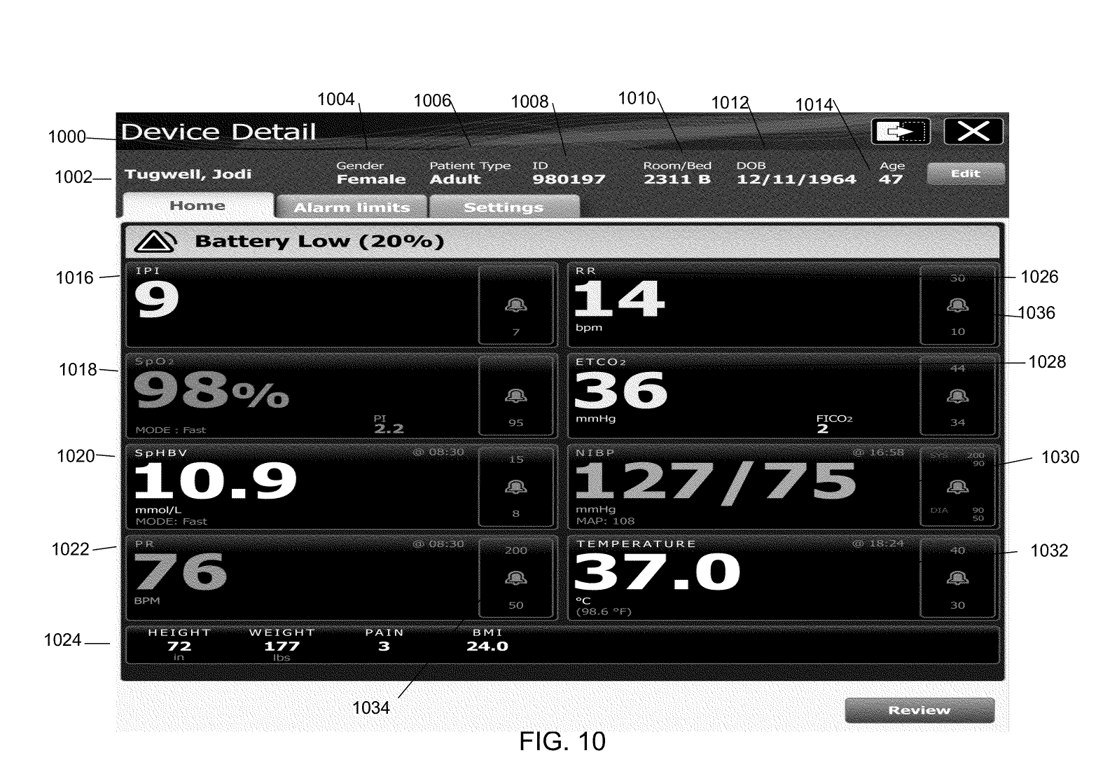

FIG. 10 shows an example screen shot 1000 of a device detail screen. The example device detail screen is displayed when a user clicks a normal display tile, for example display tile 202, on the example user interface 200. The device detail screen provides more information than can be viewed in the space of the display tile 202. For example, as shown in FIG. 10, the device detail screen shows current values of physiological parameters being measured for a patient and also shows range limits for these physiological parameters.

Information included on the example screen shot 1000 of the device detail screen includes a patient name 1002, patient gender 1004, patient type 1006 (adult or child), patient ID 1008, a room/bed number 1010 assigned to the patient, a date of birth (DOB) 1012 for the patient and an age of the patient 1014. Physiological data displayed for the patient include integrated pulmonary index (IPI) 1016, oxygen saturation (SPO2) 1018, venous calibrated total hemoglobin (SpHBV) 1020, pulse rate (PR) 1022, respiration rate (RR) 1026, end tidal carbon dioxide (ETCO2) 1028, non-invasive blood pressure (NIBP) 1030 and temperature (1032). Range limits shown include a pulse rate range limit 1034 showing an upper limit of 200 and a lower limit of 50 and a respiration rate limit 1036 showing an upper limit of 30 and a lower limit of 10. In addition, other patient information 1024 is provided including height, weight, pain index and body mass index (BMI). Additional or other patient information, physiological data and range limits are possible.

At any time after a patient is hooked up to a medical device and a compressed display tile is added to the waiting area 206 for the patient, the patient may be completely identified, for example by entering a location and/or a patient identifier for the patient. When the patient is completely identified in this manner, the compressed tile for the patient is removed from the waiting area and place in the main tile area on the user interface 200. As shown, in FIG. 2, the main tile area is an area from display tile 202 to display tile 204, typically an area of 36 tiles or 48 tiles in size.

When a compressed tile is removed from the waiting area 206, expanded to a new normal tile, and placed in the main tile area, the location at which the new tile is placed in the main tile area may be determined via one of three modes—mapped mode, manual mode and automatically sorted mode. In the mapped mode, every tile position on the main tile area is directly mapped to a known location. The location is typically determined by a unit/room/bed combination. For situations where a location does not have an assigned patient or device, an empty room tile is displayed. An empty room tile is a tile similar to the display tile shown in FIG. 6, except that the patient information area 604 and the parameter display area 606 are blank.

In the manual mode, tiles may be dragged from the waiting area to any available space in the main tile area. In automatically sorted mode, tiles are sorted according to configured criteria. For example, tiles may be sorted by room number, with the lowest room number corresponding to display tile 202 and the highest room number corresponding to display tile 204. When a tile is moved from the waiting area 206 to the main tile area using this criterion, the positions of one or more tiles in the main tile area may be adjusted to make room for a new tile. Other criteria for automatically sorting tiles are possible.

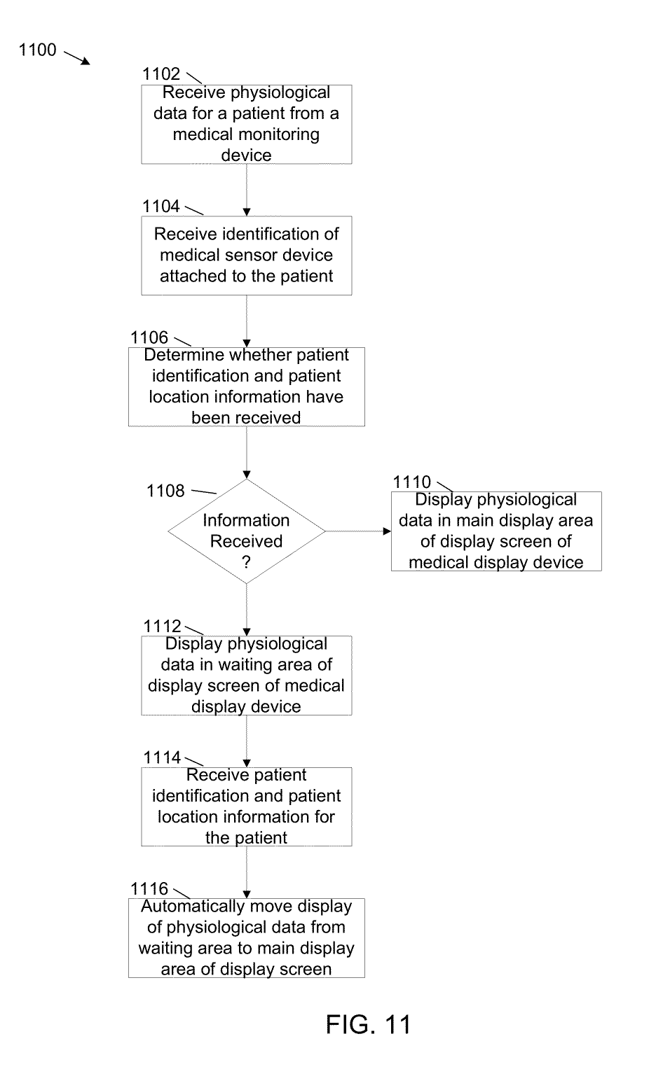

FIG. 11 shows an example flowchart for a method 1200 for entering patient data into a medical display device. The medical display device is central monitoring station, for example central monitoring station 110. At operation 1102, physiological data for a patent is received from a patient monitoring device, for example from patient monitoring device 106. Patient monitoring device 106 receives physiological data from one or more medical sensor devices, for example medical sensor devices 102, which are attached to a patient.

At operation 1104, information is received that identifies a medical sensor device that is attached to the patient. The identification information is typically received along with the physiological data that is transmitted from the medical sensor device to patient monitoring device 106. When patient monitoring device 106 transmits the physiological data to central monitoring station 110, the identification information is transmitted to central monitoring station 110 along with the physiological data. Thus, typically, whenever central monitoring station 110 receives physiological data from a medical sensor device, central monitoring station 110 receives identification information for the medical sensor device. The identification information typically includes the type of medical sensor device, for example an oxygen saturation sensor, and may also include the manufacturer of the medical sensor device and a serial number for the medical sensor device. Other identification information is possible.

At operation 1106, a determination is made as to whether patient identification and patient location information have been received for the patient. When a patient enters a medical facility under non-emergency circumstances, identification information is typically entered into a hospital database for the patient. In a hospital, the patient is typically given a wristband which contains an identifier for the patient, typically in the form of a bar code. The patient may also be assigned a room number and a bed number in the hospital. However, when a patient enters a medical facility in an emergency situation, the patient may be hooked up to medical sensor devices immediately, before the patient is given an identifier and before a room number is assigned to the patient.

When it is determined at operation 1108 that physiological data is being received for the patient and the patient identification and location information have been received for the patient, at operation 1110 the physiological data is displayed in a main display area, for example in one of display tiles 202-204 on the user interface 200 of the central monitoring station 110. The physiological data is displayed in a display tile, for example in display tile 202 in the main display area. The display tile 202 is considered to be a normal display tile in that display tile is a standard size, includes a font size that can be easily readable at normal distances, for example from a nurse's station, and includes a full complement of physiological parameters associated with the display tile.

However, when it is determined at operation 1108 that physiological data is being received for the patient but that the patient identification and location information have not been received for the patient, at operation 1112, the physiological data is displayed in a waiting area, for example waiting area 206 of a display screen, for example user interface 200 of the medical display device, for example central monitoring station 110. The physiological data is displayed in a compressed display tile. The compressed display tile is smaller than a normal display tile, has smaller font than a normal display tile and typically includes only a subset of parameters available for a normal display tile.

At operation 1114, patient identification information and patient location information are received for the patient. For example, the patient may be stabilized such that a wristband with patient identification can be created, attached to the patient and scanned by a bar code reader. The patient may also be moved out of an emergency room or intensive care unit to a standard room in the hospital so that a room and bed number can be entered for the patient.

At operation 1116, the display of physiological data in the temporary location is automatically moved to the main display area of the display screen. For example, compressed tile 210 in waiting area 206 is automatically moved to a tile location in the main display area, for example display tile 204 location, when it is determined that patient identification information and patient location information have been received for the patient.

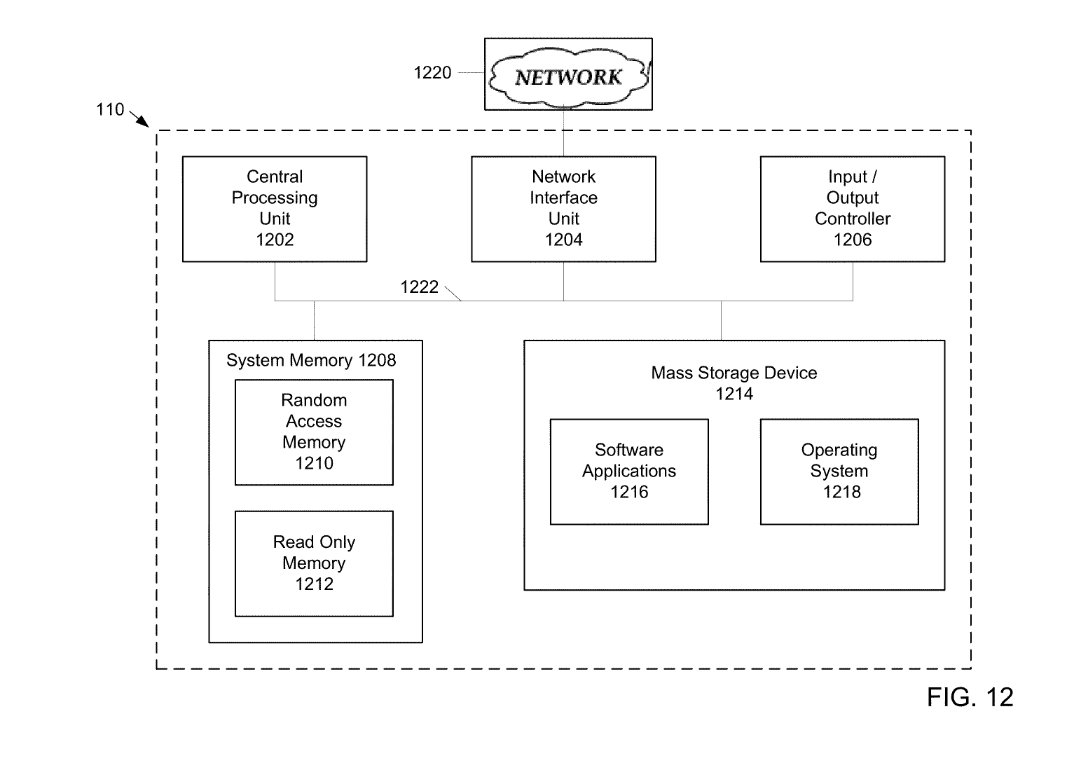

FIG. 12 illustrates example physical components of the central monitoring station 110. As illustrated in the example of FIG. 12, the central monitoring station 110 includes at least one central processing unit (“CPU”) 1202, a system memory 1208, and a system bus 1222 that couples the system memory 1208 to the CPU 1202. The system memory 1208 includes a random access memory (“RAM”) 1210 and a read-only memory (“ROM”) 1212. A basic input/output system contains the basic routines that help to transfer information between elements within the central monitoring station 110, such as during startup, is stored in the ROM 1212. The central monitoring station 110 further includes a mass storage device 1214. The mass storage device 1214 is able to store software instructions and data.

The mass storage device 1214 is connected to the CPU 1202 through a mass storage controller (not shown) connected to the bus 1222. The mass storage device 1214 and its associated computer-readable data storage media provide non-volatile, non-transitory storage for the central monitoring station 110. Although the description of computer-readable data storage media contained herein refers to a mass storage device, such as a hard disk or CD-ROM drive, it should be appreciated by those skilled in the art that computer-readable data storage media can be any available non-transitory, physical device or article of manufacture from which the central monitoring station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the central monitoring station 110.

According to various embodiments of the invention, the central monitoring station 110 may operate in a networked environment using logical connections to remote network devices through the network 1220, such as a local network, the Internet, or another type of network. The central monitoring station may connect to the network 1220 through a network interface unit 1204 connected to the bus 1222. It should be appreciated that the network interface unit 1204 may also be utilized to connect to other types of networks and remote computing systems. The central monitoring station 110 also includes an input/output controller 1206 for receiving and processing input from a number of other devices, including a keyboard, a mouse, a touch user interface display screen, or another type of input device. Similarly, the input/output controller 1206 may provide output to a touch user interface display screen, a printer, or other type of output device.

As mentioned briefly above, the mass storage device 1214 and the RAM 1210 of the central monitoring station 110 can store software instructions and data. The software instructions include an operating system 1218 suitable for controlling the operation of the central monitoring station 110. The mass storage device 1214 and/or the RAM 1210 also store software instructions, that when executed by the CPU 1202, cause the central monitoring station 110 to provide the functionality of the central monitoring station 110 discussed in this document. For example, the mass storage device 1214 and/or the RAM 1210 can store software instructions that, when executed by the CPU 1202, cause the central monitoring station 110 to display the user interface 200 and other screens.

The description of the example physical components used on the central monitoring station 110 as shown in FIG. 12 also applies to example physical components used in the EMR system 114. Thus, each of the one or more computing devices in the EMR system 114 includes at least one central processing unit (“CPU”), a system memory, and a system bus that couples the system memory to the CPU. The system memory also includes a random access memory (“RAM”), a read-only memory (“ROM”) and a mass storage device that is able to store software instructions and data. In addition, the mass storage device and its associated computer-readable data storage media provide non-volatile, non-transitory storage for each of the one or more computing devices in the EMR system 114.

The various embodiments described above are provided by way of illustration only and should not be construed to limiting. Various modifications and changes that may be made to the embodiments described above without departing from the true spirit and scope of the disclosure.

What is claimed is:

1. A method for displaying patient data, the method comprising:
receiving physiological data from a medical monitoring device, the medical monitoring device obtaining the physiological data from a patient;
determining whether patient identification information and room location information have been received for the patient; and
when it is determined that both the patient identification information and the room location information for the patient have not been received, displaying the physiological data at a temporary location on a display screen of a medical display device.

2. The method of claim 1, further comprising:
receiving the patient identification information and the room location information; and
when the patient identification information and the room location information have been received, moving a display of physiological data for the patient from the temporary location to a main display area on the display screen.

3. The method of claim 2, wherein the main display area includes a plurality of first display tiles, the first display tiles being displayed on the main display area in a grid of rows and columns.

4. The method of claim 3, wherein the physiological data for the patient is displayed at the temporary location in a second display tile, the second display tile displaying a subset of physiological data available for display on the first display tiles.

5. The method of claim 4, wherein the second display tile displays only one of continuous physiological data and episodic physiological data.

6. The method of claim 4, wherein a display font for the second display tile is smaller than for the first display tiles.

7. The method of claim 4, wherein the second display tile is displayed over the first display tiles, partially obscuring one or more of the first display tiles.

8. The method of claim 4, further comprising, when the display of physiological data for the patient is moved from the temporary location to the main display area, the second display tile is converted to a format of a first display tile, removed from the temporary location and displayed in the format of the first display tile.

9. The method of claim 4, further comprising, when the display of physiological data for the patient is moved from the temporary location to the main display area, automatically mapping the second display tile to a position in the main display area.

10. The method of claim 4, further comprising, when the display of physiological data for the patient is moved from the temporary location to the main display area, automatically placing the second display tile in a preconfigured position in the main display area.

11. The method of claim 1, further comprising receiving physiological data from one or more additional patients for whom both the patient identification information and the room location information have not been received, the physiological data from the one or more additional patients being displayed in the temporary location on the display screen of the medical display device.

12. The method of claim 11, wherein the physiological data for each of the one or more additional patients is displayed in the temporary location in a display tile, the temporary location growing in size as each display tile is added.

13. The method of claim 1, wherein the medical display device is a central monitoring station that receives physiological data from a plurality of medical monitoring devices.

14. An electronic computing device comprising:
a processing unit; and
system memory, the system memory including instructions that when executed by the processing unit cause the electronic computing device to:
receive physiological data from a medical monitoring device, the medical monitoring device being connected to a medical sensor device that is attached to a patient;
determine whether patient identification information and room location information have been received for the patient; and
when it is determined that both the patient identification information and the room location information for the patient have not been received, display the physiological data at a temporary location on a display screen of the electronic computing device.

15. The electronic computing device of claim 14, wherein the display screen of the electronic computing device includes a main display area, the main display area displaying a plurality of first display tiles, the first display tiles being displayed in the main display area in a grid of rows and columns.

16. The electronic computing device of claim 15, wherein the physiological data for the patient is displayed at the temporary location in a second display tile, the second display tile being a compressed display tile, the second display tile including a subset of physiological data available for display on the first display tiles.

17. The electronic computing device of claim 16, wherein the second display tile is displayed over the first display tiles, partially obscuring one or more of the first display tiles.

18. The electronic computing device of claim 16, wherein when it is determined that both the patient identification information and the room location information for the patient have been received, the second display tile is converted to a format of a first display tile, removed from the temporary location and displayed in the main display area in the format of the first display tile.

19. The electronic computing device of claim 14, wherein the electronic computing device is a central monitoring station that receives physiological data from a plurality of medical monitoring devices.

20. A computer-readable data storage medium comprising instructions that, when executed by a processing unit of an electronic computing device, cause the electronic computing device to:
display physiological data for a plurality of patients in a main display area of a display screen of the electronic computing device, the physiological data for the plurality of patients being displayed as first display tiles in the main display area, the physiological data for each of the plurality of patients being displayed in a different first display tile, the first display tiles being oriented in rows and columns on the main display area;
receive physiological data for an additional patient from a medical monitoring device, the additional patient being a new patient, the medical monitoring device being connected to a medical sensor device that is attached to the new patient;
determine whether patient identification information and room location information have been received for the new patient;
when it is determined that both the patient identification information and the room location information have not been received for the new patient, display the physiological data for the new patient at a temporary location in the main display area of the electronic computing device, the physiological data for the new patient being displayed in a second display tile at the temporary location, the second display tile partially obscuring one or more first display tiles, the second display tile being a compressed version of a first display tile, the second display tile displaying a subset of physiological data available for display at the first display tile, the second display tile displaying the physiological data in a smaller font size than used for the first display tile;
receive the patient identification information and the room location information for the new patient; and
when the patient identification information and the room location information have been received for the new patient:
automatically convert the second display tile to a format of the first display tile;
automatically remove the second display tile from the temporary location; and
automatically relocate the second display tile to the main display area.
